(19) United States
(12) Patent Application Publication
(10) Pub. No.: US 2019/0011444 A1
(43) Pub. Date: Jan. 10, 2019

(54) SENSING METHOD

(71) Applicant: SIWARD Crystal Technology Co., Ltd., Taichung City (TW)

(72) Inventors: Chuen-Yuan Hsu, Taichung City (TW); Yi-Heui Hsieh, Taichung City (TW); Chiao-Wen Huang, Taichung City (TW); Ting Liu, Taichung City (TW)

(21) Appl. No.: 15/915,161

(22) Filed: Mar. 8, 2018

(30) Foreign Application Priority Data
Jul. 6, 2017 (TW) 106122747

(51) Int. Cl. G01N 33/543; G01N 21/552

(52) U.S. Cl. CPC G01N 33/54373; G01N 21/554

(57) ABSTRACT

A sensing method is disclosed. The sensing method includes steps of providing a carrier including a hole having a bottom, wherein a plurality of spaced apart first nanoparticles are disposed on the bottom; coating a sensing molecule in the hole; providing a testing solution having a testing parameter to the hole, wherein the testing solution has a complex including a testing molecule and a second nanoparticle, and a specific binding occurs between the testing molecule and the sensing molecule; centrifuging the carrier to subside the complex; washing the hole; and measuring a synthetic spectral signal change of the first nanoparticle and the second nanoparticle according to a degree of the specific binding between the testing molecule and the sensing molecule to determine the testing parameter of the testing solution.

### CROSS REFERENCE TO RELATED APPLICATIONS

The application claims the benefit of Taiwan Patent Application No. 106122747, filed on Jul. 6, 2017, at the Taiwan Intellectual Property Office, the disclosures of which are incorporated herein in their entirety by reference.

### FIELD OF THE INVENTION

The present invention is related to a sensing method, and more particularly to a sensing method using localized surface plasmon resonance.

### BACKGROUND OF THE INVENTION

The principle of localized surface plasmon resonance is that when metal nanoparticles are fabricated on a transparent substrate, the excitation of the incident light will cause the nanoparticles' surface to produce surface plasmonic resonance. Because the frequency and intensity of the resonance are susceptible to the surrounding environment and produce a wavelength shift or a variation of intensity of a signal, the change of the local dielectric constant can be used to detect analytes. As long as there are analytes binding adjacent to the particles, an optical variation can be measured by an optical instrument.

However, the penetration depth of the plasmon field for localized surface plasmon resonance is between 15-30 nm. Hence, localized surface plasmon resonance is less susceptible to effects occurring away from the surface. In other words, localized surface plasmon resonance detects changes very close to the surface. Therefore, it is an urgent goal for the skilled person in the art to further enhance the sensitivity of localized surface plasmon resonance.

### SUMMARY OF THE INVENTION

In accordance with one aspect of the present invention, a sensing method is disclosed. The sensing method includes steps of providing a carrier having a hole including a bottom, wherein a substrate is disposed on the bottom of the hole, a plurality of first nanoparticles are disposed on the substrate and spaced apart from each other, and the plurality of first nanoparticles generate a first spectral signal when excited; providing a first organic molecule into the hole; coating the first organic molecule in the hole; providing a second organic molecule into the hole, wherein when the second organic molecule has a first specific binding with the first organic molecule, the first spectral signal converts to a second spectral signal if the plurality of first nanoparticles are excited; providing a complex including a third organic molecule into the hole; centrifuging the carrier to subside the complex, wherein when the third organic molecule has a second specific binding with the second organic molecule, the second specific binding amplifies the second spectral signal into a third spectral signal having a specific value if the plurality of first nanoparticles are excited, wherein the first, the second and the third spectral signals are generated due to localized surface plasmon resonance; and measuring the specific value of the third spectral signal.

In accordance with another aspect of the present invention, a sensing method is disclosed. The sensing method includes steps of providing a carrier including a hole having a bottom disposed a substrate thereat wherein a plurality of first nanoparticles are disposed on the substrate and spaced apart from each other, and the plurality of first nanoparticles generate a spectral signal when excited; providing a plurality of first molecules into the hole; coating the first molecules in the hole; providing a plurality of complexes including a plurality of second molecules and a plurality of second nanoparticles into the hole; centrifuging the carrier to subside the complex, wherein: when the second molecules have a specific binding with the first molecules, the spectral signal generated by the plurality of first nanoparticles produces a change having a value; and when the second molecules have the specific binding with the first molecules, a coupling effect is generated between the first nanoparticles and the second nanoparticles to amplify the change of the spectral signal; and measuring the value of the change, wherein the change is produced through localized surface plasmon resonance.

In accordance with a further aspect of the present invention, a sensing method is disclosed. The sensing method includes steps of providing a carrier including a hole having a bottom, wherein a plurality of spaced apart first nanoparticles are disposed on the bottom; coating a sensing molecule in the hole; providing a testing solution having a testing parameter to the hole, wherein the testing solution has a complex including a testing molecule and a second nanoparticle, and a specific binding occurs between the testing molecule and the sensing molecule; centrifuging the carrier to subside the complex; washing the hole; and measuring a synthetic spectral signal change of the first nanoparticle and the second nanoparticle according to a degree of the specific binding between the testing molecule and the sensing molecule to determine the testing parameter of the testing solution.

### BRIEF DESCRIPTION OF THE DRAWINGS

FIG. 1 shows the sensing method according to the first embodiment of the present invention;

FIG. 2 shows the sensing method according to the second embodiment of the present invention;

FIG. 3 shows the sensing method according to the third embodiment of the present invention;

FIG. 4 shows a spectrum of gold nanoparticles of the present invention;

FIG. 5 shows a normalized spectrum of complexes of the horseradish peroxidase-streptavidin and gold nanoparticles (SA-HRP@gold nanoparticles) of the present invention;

FIG. 6 shows a spectrum of carrying out localized surface plasmon resonance experiments after standing or centrifuging complexes of goat anti-rabbit immunoglobulin G conjugated horseradish peroxidase and gold nanoparticles (anti-rabbit IgG-HRP@Au nanoparticles) of the present invention;

FIG. 7 shows a spectrum of the influence of centrifugal spin-dry biotin conjugated goat anti-rabbit immunoglobulin G (anti-rabbit IgG-biotin) or not on the detection sensitivities of the experiments of localized surface plasmon resonance of the present invention;

FIG. 8 shows a spectrum of the sensitivities of experiments of localized surface plasmon resonance in the condition of carrying out centrifugal sedimentation after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles) of the present invention;

FIG. 9 shows a spectrum of the sensitivities of experiments of localized surface plasmon resonance in the condition of standing after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles) of the present invention;

FIG. 10 shows a spectrum of the sensitivities of experiments of localized surface plasmon resonance of carrying out centrifugal sedimentation only after adding complexes of horseradish peroxidase-streptavidin and gold nanoparticles (SA-HRP@gold nanoparticles) of the present invention;

FIG. 11 shows a spectrum of the sensitivities of experiments of localized surface plasmon resonance of carrying out centrifugal sedimentation after adding biotin-conjugated goat anti-rabbit immunoglobulin G (anti-rabbit IgG-biotin) and complexes of horseradish peroxidase-streptavidin and gold nanoparticles (SA-HRP@gold nanoparticles) of the present invention;

FIG. 12 shows a spectrum of the sensitivities of experiments of localized surface plasmon resonance of carrying out centrifugal sedimentation after adding blocking buffer and complexes of horseradish peroxidase-streptavidin and gold nanoparticles (SA-HRP@gold nanoparticles) of the present invention;

FIG. 13 shows a spectrum of the sensitivities of experiments of localized surface plasmon resonance of carrying out centrifugal sedimentation after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles) of the present invention;

FIG. 14 shows a plot of the absorbance at 550 nm of the spectrum of the sensitivities of experiments of localized surface plasmon resonance measured after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles) versus the concentration of biotin conjugated goat anti-rabbit immunoglobulin G of the present invention;

FIG. 15 shows a spectrum of the sensitivities of experiments of localized surface plasmon resonance for detection of hepatitis C virus antibodies in the serum of a patient with hepatitis C in the condition of standing after adding the patient's serum of the present invention;

FIG. 16 shows a spectrum of the sensitivities of experiments of localized surface plasmon resonance for detection of hepatitis C virus antibodies in the serum of the patient with hepatitis C in the condition of centrifuging after adding the patient's serum of the present invention; and

FIG. 17 shows a plot of the absorbance at 550 nm of the spectrum of the sensitivities of experiments of localized surface plasmon resonance for detection of hepatitis C virus antibodies in the serum of the patient with hepatitis C in the condition of centrifuging after adding the patient's serum versus concentration of dilution fold of the patient's serum of the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

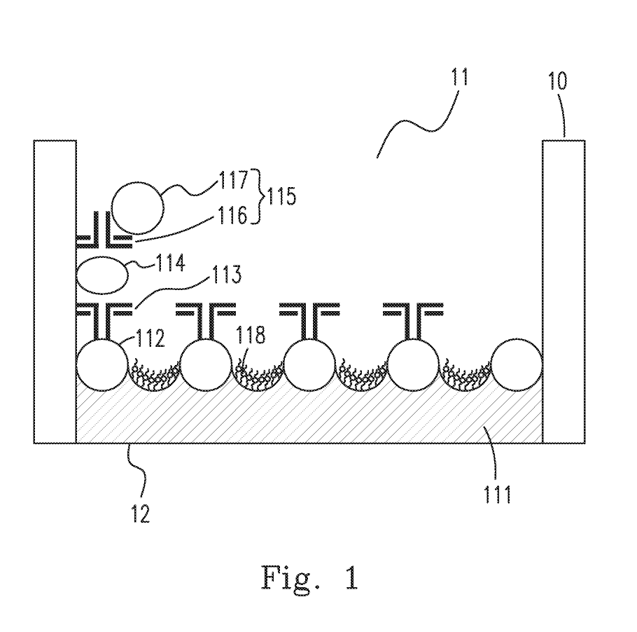

Please refer to FIG. 1, wherein the first embodiment of the present invention discloses a sensing method. The sensing method includes the following steps. Provide a carrier 10 having a hole 11 including a bottom 12, wherein a substrate 111 is disposed on the bottom 12 of the hole 11, a plurality of first nanoparticles 112 are disposed on the substrate 111 and spaced apart from each other, and the plurality of first nanoparticles 112 generate a first spectral signal when excited; provide a first organic molecule 113 into the hole 11; coat the first organic molecule 113 in the hole 11; provide a second organic molecule 114 into the hole 11, wherein when the second organic molecule 114 has a first specific binding with the first organic molecule 113, the first spectral signal converts to a second spectral signal if the plurality of first nanoparticles 112 are excited; provide a complex 115 including a third organic molecule 116 into the hole 11; centrifuge the carrier 10 to subside the complex 115, wherein when the third organic molecule 116 has a second specific binding with the second organic molecule 114, the second specific binding amplifies the second spectral signal into a third spectral signal having a specific value if the plurality of first nanoparticles 112 are excited, wherein the first, the second and the third spectral signals are generated due to localized surface plasmon resonance; and measure the specific value of the third spectral signal.

The carrier 10 in this embodiment may be but is not limited to a strip, a plate, or a microchannel device such as a microchannel chip. When the carrier 10 is the strip, the strip can be combined with a frame to facilitate centrifugation and measurement. The substrate 111 is made of a transparent material, the transparent material may be but is not limited to a glass or a plastic. Each of the first nanoparticle 112 may include a metal, and the metal may be at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir). A silane 118 may be coated between the plurality of first nanoparticles 112 on the substrate 111. The silane 118 may be an alkylsilane, aminosilane or other silanes. The aminosilane may be but is not limited to a 3-aminopropryltrimethoxysilane (APTMS) or 3-aminopropyltriethoxysilane (APTES).

The first organic molecule 113 may be but is not limited to an antigen or an antibody. The way to coat the first organic molecule 113 may be but is not limited to standing or microwaving, and the condition for standing may be but is not limited to overnight (O/N) at 4° C., and the condition for microwaving may be but is not limited to 60 W, 30-40 minutes.

A blocking buffer may be provided into the hole 11 after coating the first organic molecule 113, the carrier 10 may be centrifuged to block the area where the first organic molecule 113 is not coated after providing the blocking buffer into the hole 11. The centrifuging step may be but is not limited to be performed under 500-6000 rpm for 15-60 minutes or 2000-3000 rpm for 15-60 minutes.

The second organic molecule 114 may be but is not limited to an antigen or an antibody. The second organic molecule 114 may include a second organic sub-molecule, and the second organic molecule may be a biotin. After providing the second organic molecule 114 into the hole 11, the carrier 10 may be centrifuged to subside the second organic molecule 114 to the bottom 12 of the hole 11. The centrifuging step may be but is not limited to be performed under 500-6000 rpm for 15-60 minutes or 2000-3000 rpm for 15-60 minutes. The second organic molecule 114 may be present in a testing solution, and the testing solution may be but is not limited to an experimental sample or a specimen. The specimen may be but is not limited to blood, urine, cell culture media and other body fluids.

When the second organic molecule 114 has the first specific binding with the coated first organic molecule 113, a light absorption rate of the first nanoparticles 112 increases, and thus the first spectral signal converts to a second spectral signal if the plurality of first nanoparticles are excited.

The third organic molecule 116 may be but is not limited to an antigen or an antibody, any organic molecule having a specific binding force with the second organic molecule 114 or the second organic sub-molecule may be used. For example, the third organic molecule 116 may be a streptavidin.

The complex further includes a second nanoparticle 117 coupled to the third organic molecule 116. Each of the second nanoparticle 117 includes a metal, and the metal is at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir).

When the third organic molecule 116 has a second specific binding with the second organic molecule 114 or the second organic sub-molecule, the distance between the first nanoparticle 112 and the second nanoparticle 117 becomes closer, and thus a coupling effect between the first nanoparticle 112 and the second nanoparticle 117 generates, which is a dipolar coupling effect. Therefore, the second spectral signal is amplified to be the third spectrum signal if the plurality of first nanoparticles are excited.

In the sensing method in this embodiment, the first organic molecule 113, the second organic molecule 114, and the complex 115 may be all of a liquid form. The sensing method may further comprise the following steps. Place the carrier 10 in a rotor of a centrifuge and centrifuge the carrier 10 to subside the complex 115. The centrifuging step may be but is not limited to be performed under 500-6000 rpm for 15-60 minutes or 2000-3000 rpm for 15-60 minutes. Turn the carrier 10 by 180 degrees so that the hole 11 faces a bottom of a rotor; and centrifuge the carrier 10 to remove a liquid in the hole 11. The centrifuging step may be but is not limited to be performed under 500-6000 rpm for 0.5-2 minutes or 2700 rpm for 0.5-2 minutes. In the sensing method in this embodiment, the change may be measured from a full wavelength or a single wavelength. The single wavelength may be 550 nm.

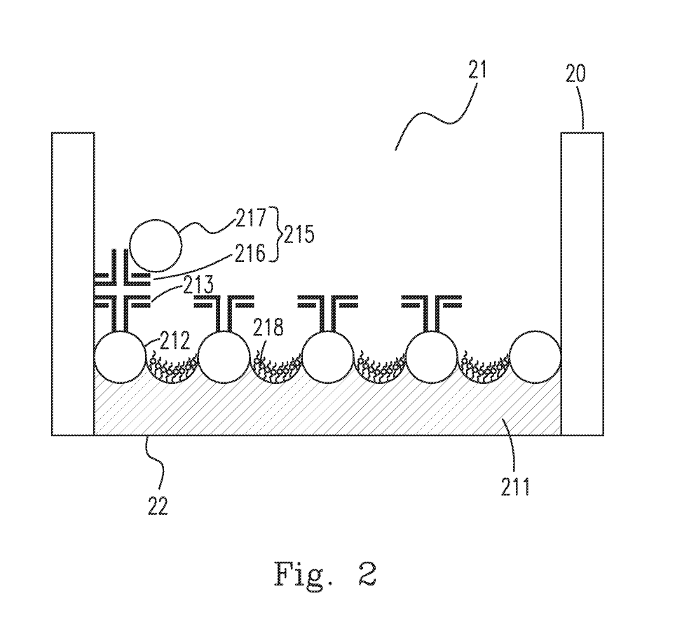

Please refer to FIG. 2, wherein the second embodiment of the present invention discloses a sensing method. The sensing method includes the following steps. Provide a carrier 20 including a hole 21 having a bottom 22 disposed a substrate 211 thereat, wherein a plurality of first nanoparticles 212 are disposed on the substrate 211 and spaced apart from each other, and the plurality of first nanoparticles 212 generate a spectral signal when excited; provide a plurality of first molecules 213 into the hole 21; coat the first molecules 213 in the hole; provide a plurality of complexes 215 including a plurality of second molecules 216 and a plurality of second nanoparticles 217 into the hole; centrifuge the carrier 20 to subside the complex 215, wherein when the second molecules 216 have a specific binding with the first molecules 213, the spectral signal generated by the plurality of first nanoparticles 212 produces a change having a value, and when the second molecules 216 have the specific binding with the first molecules 213, a coupling effect is generated between the first nanoparticles 212 and the second nanoparticles 217 to amplify the change of the spectral signal; and measure the value of the change, wherein the change is produced through localized surface plasmon resonance.

The carrier 20 in this embodiment may be but is not limited to a strip, a plate, or a microchannel device such as a microchannel chip. When the carrier 20 is the strip, the strip can be combined with a frame to facilitate centrifugation and measurement. The substrate 211 is made of a transparent material, the transparent material may be but is not limited to a glass or a plastic. Each of the first nanoparticle 212 may include a metal, and the metal may be at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir). A silane 218 may be coated between the plurality of first nanoparticles 212 on the substrate 211. The silane 218 may be an alkylsilane, aminosilane or other silane. The aminosilane may be but is not limited to a 3-aminopropryltrimethoxysilane (APTMS) or 3-aminopropyltriethoxysilane (APTES).

The first molecule 213 may be but is not limited to an antigen or an antibody. The way to coat the first molecule 213 may be but is not limited to standing or microwaving, and the condition for standing may be but is not limited to overnight (O/N) at 4° C., the condition for microwaving may be but is not limited to 60 W, 30-40 minutes.

A blocking buffer may be provided into the hole 21 after coating the first molecule 213, the carrier 20 may be centrifuged to block the area where the first molecule 213 is not coated after providing the blocking buffer into the hole 21. The centrifuging step may be but is not limited to be performed under 500-6000 rpm for 15-60 minutes or 2000-3000 rpm for 15-60 minutes.

The second molecule 216 may be but is not limited to an antigen or an antibody. Each of the second nanoparticle 217 includes a metal, and the metal is at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir). The centrifuging step for the carrier 20 may be but is not limited to be performed under 500-6000 rpm for 15-60 minutes or 2000-3000 rpm for 15-60 minutes.

When the second molecule 216 has the specific binding with the coated first molecule 213, the light absorption rate of the first nanoparticles 212 increases, and thus the spectral signal generates the change. And, when the second molecule 216 has the specific binding with the coated first molecule 213, the distance between the first nanoparticle 212 and the second nanoparticle 217 becomes closer, and thus the coupling effect between the first nanoparticle 212 and the second nanoparticle 217 generates, which is a dipolar coupling effect. Therefore, the change generated by the spectrum signal is amplified.

In the sensing method in this embodiment, the first molecule 213 and the complex 115 may be both of a liquid form. The sensing method may further comprise the following steps. Place the carrier 20 in a rotor of a centrifuge and centrifuge the carrier 20 to subside the complex 215. The centrifuging step may be but is not limited to be performed under 500-6000 rpm for 15-60 minutes or 2000-3000 rpm for 15-60 minutes. Turn the carrier 20 by 180 degrees so that the hole 21 faces a bottom of a rotor; and centrifuge the carrier 20 to remove a liquid in the hole 21. The centrifuging step may be but is not limited to be performed under 500-6000 rpm for 0.5-2 minutes or 2700 rpm for 0.5-2 minutes. In the sensing method in this embodiment, the change may be measured from a full wavelength or a single wavelength. The single wavelength may be 550 nm.

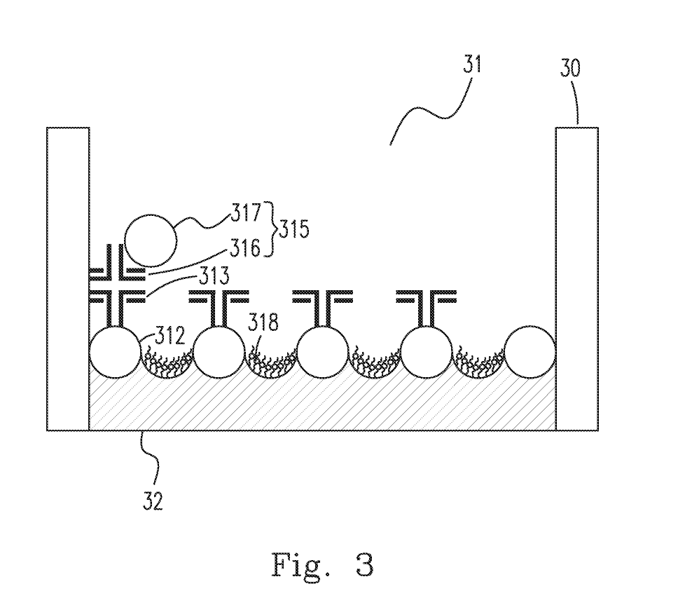

Please refer to FIG. 3, wherein the third embodiment of the present invention discloses a sensing method. The sensing method includes the following steps. Provide a carrier 30 including a hole 31 having a bottom 32, wherein a plurality of spaced apart first nanoparticles 312 are disposed on the bottom 32; coat a sensing molecule 313 in the hole 31; provide a testing solution having a testing parameter to the hole 31, wherein the testing solution has a complex 315 including a testing molecule 316 and a second nanoparticle 317, and a specific binding occurs between the testing molecule 316 and the sensing molecule 313; centrifuge the carrier 30 to subside the complex 315; wash the hole 31; and measure a synthetic spectral signal change of the first nanoparticle 312 and the second nanoparticle 317 according to a degree of the specific binding between the testing molecule 316 and the sensing molecule 313 to determine the testing parameter of the testing solution.

The carrier 30 in this embodiment may be but is not limited to a strip, a plate, or a microchannel device such as a microchannel chip. When the carrier 30 is the strip, the strip can be combined with a frame to facilitate centrifugation and measurement. Each of the first nanoparticle 312 may include a metal, and the metal may be at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir). A silane 318 may be coated between the plurality of first nanoparticles 312. The silane 318 may be an alkylsilane, aminosilane or other silane. The aminosilane may be but is not limited to a 3-aminopropryltrimethoxysilane (APTMS) or 3-aminopropyltriethoxysilane (APTES).

The sensing molecule 313 may be but is not limited to an antigen or an antibody. The way to coat the sensing molecule 313 may be but is not limited to standing or microwaving, and the conditions for standing may be but is not limited to overnight (O/N) at 4° C., the conditions for microwaving may be but is not limited to 60 W, 30-40 minutes.

A blocking buffer may be provided into the hole 31 after coating the sensing molecule 313, the carrier 30 may be centrifuged to block the area where the sensing molecule 313 is not coated after providing the blocking buffer into the hole 31. The centrifuging step may be but is not limited to be performed under 500-6000 rpm for 15-60 minutes or 2000-3000 rpm for 15-60 minutes.

The testing solution may be but is not limited to an experimental sample or a specimen. The specimen may be but is not limited to blood, urine, cell culture media and other body fluids. The testing molecule 316 may be but is not limited to an antigen or an antibody. Each of the second nanoparticle 317 may include a metal, and the metal may be at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir). After providing the complex 315 to the hole 31, the centrifuging step for the carrier 30 may be but is not limited to be performed under 500-6000 rpm for 15-60 minutes or 2000-3000 rpm for 15-60 minutes.

When the testing molecule 316 has a specific binding with the coated sensing molecule 313, the light absorption rate of the first nanoparticles 312 increases. When the testing molecule 316 has the specific binding with the coated sensing molecule 313, the distance between the first nanoparticles 312 and the second nanoparticle 317 becomes closer, and thus a coupling effect between the first nanoparticles 312 and the second nanoparticle 317 generates, which is a dipolar coupling effect. Therefore, the synthetic spectral signal change of the first nanoparticles 312 and the second nanoparticle 317 occurs. The synthetic spectral signal change comes from localized surface plasmon resonance. The testing parameter may be but is not limited to a concentration of the testing molecule 316.

In the sensing method in this embodiment, the sensing molecule 313 and the complex 315 may be both of a liquid form. The sensing method may further comprise the following steps. Place the carrier 30 in a rotor of a centrifuge and centrifuge the carrier 30 to subside the complex 315. The centrifuging step may be but is not limited to performing under 500-6000 rpm for 15-60 minutes or 2000-3000 rpm for 15-60 minutes. Turn the carrier 30 by 180 degrees so that the hole 31 faces a bottom of a rotor, and centrifuge the carrier 30 to remove a liquid in the hole 31. The centrifuging step may be but is not limited to performing under 500-6000 rpm for 0.5-2 minutes or 2700 rpm for 0.5-2 minutes. In the sensing method in this embodiment, the change may be measured from a full wavelength or a single wavelength. The single wavelength may be 550 nm.

The sensing method of the present invention can reduce the overall detection time and improve the sensitivity by centrifugal sedimentation. The reason is that the traditional standing method causes molecules to be adsorbed slowly on a side wall and a bottom of a hole of a carrier, and centrifugal sedimentation can efficiently adsorb molecules on the bottom of the hole of the carrier. In particular, molecules coupled with second nanoparticles are more efficiently transported by centrifugal force to the bottom of the carrier because of their larger size and heavier weight, and more efficiently and specifically bonded with the coated molecules on the bottom, and make first nanoparticles on the bottom efficiently sense the specific binding due to the proximity and amplify the spectral signal. The spectral signal can be further amplified due to the dipolar coupling effect between the first nanoparticle and the second nanoparticle. Therefore, instead of the traditional standing adsorption, the combination of the centrifugal sedimentation technology and localized surface plasmon resonance can obtain a surprising sensing efficiency.

On the other hand, due to the increased sensitivity, the sensing method of the present invention monitors the relationship between the optical density (OD) intensity and the concentration at a single wavelength (for example, 550 nm) and good linearity can be measured, so that it is not necessary to measure a full wavelength, accurate results can be obtained by measuring only a single wavelength, which is more convenient to the user.

### EXPERIMENTS

1. Synthesize gold nanoparticles:

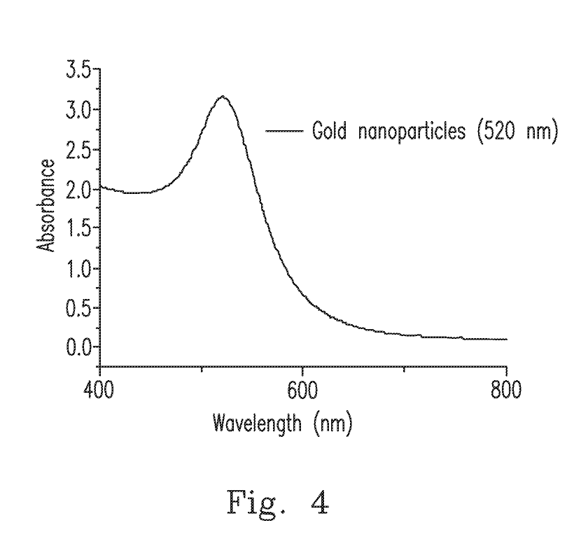

Synthesize gold nanoparticles and measure their absorption wavelength. FIG. 4 shows that the absorption wavelength of the gold nanoparticles used in the present invention is 520 nm.

2. Synthesize complexes of horseradish peroxidase-streptavidin and gold nanoparticles (SA-HRP@gold nanoparticle):

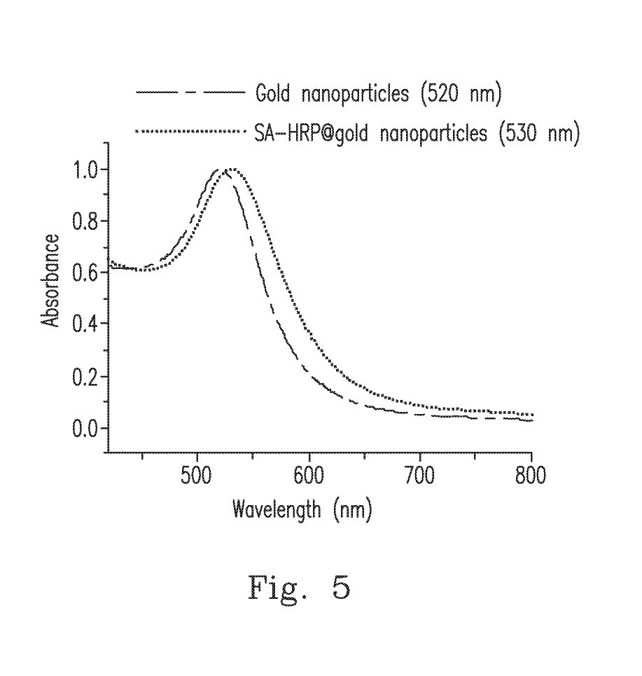

Synthesize complexes of horseradish peroxidase-streptavidin and gold nanoparticles and measure its absorption wavelength. FIG. 5 is the normalized spectrum showing that the absorption wavelength of the gold nanoparticles is 520 nm, and the absorption wavelength of complexes of horseradish peroxidase-streptavidin and gold nanoparticle shifts to 530 nm, indicating the successful adsorption of horseradish peroxidase-streptavidin onto the surfaces of the gold nanoparticles. Using this method, complexes of goat anti-rabbit immunoglobulin G conjugated horseradish peroxidase and gold nanoparticles also can be synthesized.

Use a gold nanoparticle carrier to carry out experiments of the present invention, and the gold nanoparticle carrier is a strip or a plate. When the carrier is the strip, the gold nanoparticle carrier can be combined with a frame to facilitate centrifugation and measurement. A bottom of a hole of the gold nanoparticle carrier is disposed with a substrate made of a transparent material. The substrate is disposed with a plurality of spaced apart gold nanoparticles. Silane is coated between the plurality of spaced apart gold nanoparticles.

3. Use complexes of goat anti-rabbit immunoglobulin G conjugated horseradish peroxidase and gold nanoparticles (anti-rabbit IgG-HRP@Au nanoparticles) to carry out experiments of localized surface plasmon resonance:

In order to verify whether centrifugal sedimentation can increase protein adsorption efficiency and sensitivity in experiments of localized surface plasmon resonance, carry out these experiments by using complexes of goat anti-rabbit immunoglobulin G conjugated horseradish peroxidase (Goat anti-rabbit IgG(H+L), horseradish peroxidase conjugated, abbreviated as anti-rabbit IgG-HRP) and gold nanoparticles (abbreviated as anti-rabbit IgG-HRP@Au nanoparticles) by means of standing and centrifugal sedimentation, respectively.

(1) Add 100 μL of 1500 ng/cm² rabbit immunoglobulin G (ChromPure Rabbit IgG whole molecule, abbreviated as rabbit IgG, formulated in coating buffer) into a gold nanoparticle carrier, microwave at 60 W for 30 min, and then wash with phosphate-buffered saline containing Tween 20 (Phosphate Buffered Saline with Tween 20, abbreviated as PBST) four times.

(2) Add 100 μL of 1× blocking buffer, stand at 25° C. for 2 hours, and then wash with PBST four times. After washing, turn the gold nanoparticle carrier by 180 degrees so that a hole of the gold nanoparticle carrier faces a bottom of a rotor of a centrifuge, and centrifuge the gold nanoparticle carrier at 2700 rpm for 2 minutes to remove a liquid in the hole.

(3) Add 100 μL of complexes of goat anti-rabbit immunoglobulin G conjugated horseradish peroxidase and gold nanoparticles, and stand for 1 hour and centrifuge at 2700 rpm for 30 minutes, respectively, then wash with PBST four times. After washing, turn the gold nanoparticle carrier by 180 degrees so that the hole of the gold nanoparticle carrier faces the bottom of the rotor of the centrifuge, centrifuge the gold nanoparticle carrier at 2700 rpm for 2 minutes to remove a liquid in the hole, and measure a full spectrum.

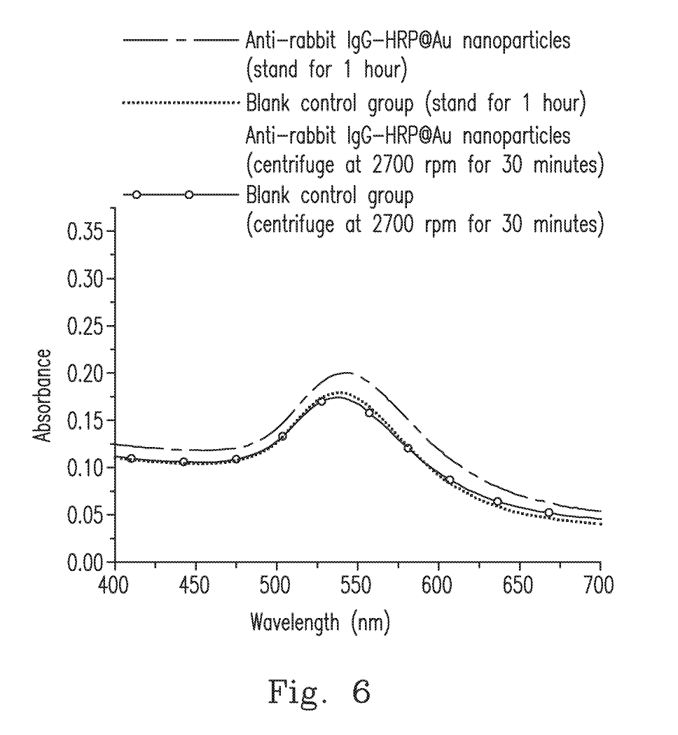

FIG. 6 and Table 1 show that by means of centrifugal sedimentation, both the spectral shift and the absorption intensity are obviously enhanced. Clearly, after binding the gold nanoparticles and goat anti-rabbit immunoglobulin G conjugated horseradish peroxidase, centrifugal sedimentation helps to improve a change of a spectral signal. Start the follow-up experiments in order to further confirm whether it is highly selective and specific.

TABLE 1

Absorbance at 550 nm

| | Stand for | Centrifuge at 2700 rpm |
|---|---|---|
| | 1 hour | for 30 minutes |
| Blank control group | 0.173 | 0.166 |
| Add anti-rabbit IgG- | 0.197 | 0.321 |
| HRP@gold nanoparticles | | |

4. The influence of centrifugal spin-dry or not on the detection sensitivity of the experiments of localized surface plasmon resonance:

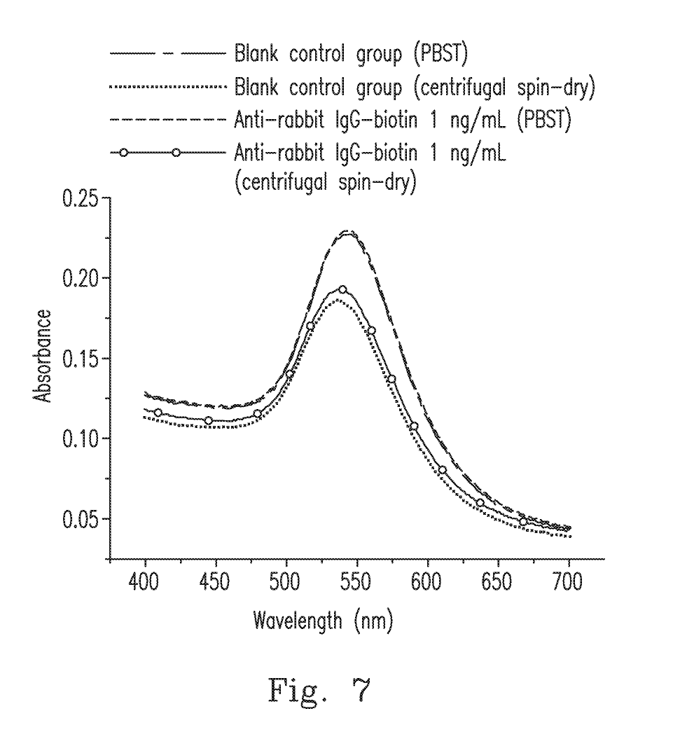

Please refer to FIG. 7. Prepare 1 ng/mL of biotin conjugated goat anti-rabbit immunoglobulin G (Biotin-SP-conjugated AffiniPure Goat Anti-Rabbit IgG(H+L), abbreviated as anti-rabbit IgG-biotin) for dry and wet environment measurement spectrum test. Measure the spectrum in a PBST environment after centrifugal sedimentation of complexes of horseradish peroxidase-streptavidin and gold nanoparticles (SA-HRP@Au nanoparticles) in a gold nanoparticle carrier. It is found that there is no difference with the blank control group.

On the contrary, measure the spectrum after pouring out a liquid and centrifugal spin-dry. It is found that there is a significant difference with the blank control group, and that centrifugal spin-dry of the gold nanoparticle carrier can improve the detection sensitivity.

5. Carry out experiments of localized surface plasmon resonance with complexes of goat anti-rabbit immunoglobulin G conjugated horseradish peroxidase and gold nanoparticles (anti-rabbit IgG-HRP@Au nanoparticles) to find out the best rotational speed of centrifugal sedimentation:

(1) Add 100 μL of 1500 ng/cm² goat anti-rabbit immunoglobulin G conjugated horseradish peroxidase (Goat anti-rabbit IgG(H+L), horseradish peroxidase conjugated, abbreviated as anti-rabbit IgG-HRP, formulated in coating buffer) into a gold nanoparticle carrier, microwave at 60 W for 30 min, and then wash with PBST four times.

(2) Add 100 μL of 1× blocking buffer, stand at 25° C. for 2 hours, and then wash with PBST four times.

(3) Add 100 μL of 10×, 50×, 250×, 1250× and 6250× diluted rabbit immunoglobulin G (ChromPure Rabbit IgG, whole molecule, abbreviated as rabbit IgG) (diluted in a blocking buffer, 1.19 mg/ml as 1×), and blank control group only add blocking buffer. Stand at 25° C. for 1 hour, and then wash with PBST four times. After washing, turn the gold nanoparticle carrier by 180 degrees so that the hole of the gold nanoparticle carrier faces a bottom of a rotor of a centrifuge, centrifuge the gold nanoparticle carrier at 2700 rpm for 2 minutes to remove a liquid in the hole, and measure a full spectrum.

(4) Add 100 μL of complexes of goat anti-rabbit immunoglobulin G conjugated horseradish peroxidase (Goat anti-rabbit IgG(H+L), horseradish peroxidase conjugated) and gold nanoparticles (abbreviated as anti-rabbit IgG-HRP@gold nanoparticles) with an absorbance of 1, and with centrifugal sedimentation at 2000 rpm and 2700 rpm for 15 minutes, respectively. After centrifugation, wash with PBST four times. After washing, turn the gold nanoparticle carrier by 180 degrees so that the hole of the gold nanoparticle carrier faces a bottom of a rotor of a centrifuge, centrifuge the gold nanoparticle carrier at 2700 rpm for 2 minutes to remove a liquid in the hole, and measure a full spectrum.

Table 2 shows that increasing the centrifugal speed to 2700 rpm can increase the absorption intensity.

TABLE 2

| | | | Centrifugal sedimentation | | Centrifugal sedimentation | |
|---|---|---|---|---|---|---|
| | | | at 2000 rpm | | at 2700 rpm | |
| | | | | The absorbance | | The absorbance |
| | | The absorbance | The peak of the | of the peak of the | The peak of the | of the peak of the |
| Dilution | The peak of the | of the peak of the | spectral absorption | spectral absorption | spectral absorption | spectral absorption |
| fold of | spectral absorption | spectral absorption | curve after adding | curve after adding | curve after adding | curve after adding |
| rabbit | curve after adding | curve after adding | anti-rabbit IgG-HRP | anti-rabbit IgG-HRP | anti-rabbit IgG-HRP | anti-rabbit IgG-HRP |
| IgG | rabbit IgG | rabbit IgG | @gold nanoparticles | @gold nanoparticles | @gold nanoparticles | @gold nanoparticles |
| 10 X | 543 | 0.194 | 568 | 0.365 | 569 | 0.421 |
| 50 X | 543 | 0.193 | 569 | 0.346 | 571 | 0.401 |
| 250 X | 542 | 0.190 | 569 | 0.349 | 575 | 0.405 |
| 1250 X | 543 | 0.191 | 570 | 0.344 | 572 | 0.409 |
| 6250 X | 541 | 0.186 | 554 | 0.239 | 569 | 0.360 |
| 0 | 540 | 0.186 | 541 | 0.182 | 541 | 0.186 |

6. Carry out experiments of localized surface plasmon resonance with complexes of goat anti-rabbit immunoglobulin G conjugated horseradish peroxidase and gold nanoparticles (anti-rabbit IgG-HRP@Au nanoparticles) to find out the best time of centrifugal sedimentation:

(1) Add 100 μL of 1500 ng/cm² goat anti-rabbit immunoglobulin G conjugated horseradish peroxidase (Goat anti-rabbit IgG(H+L), horseradish peroxidase conjugated, abbreviated as anti-rabbit IgG-HRP, formulated in coating buffer) into a gold nanoparticle carrier, microwave at 60 W for 30 min, and then wash with PBST four times.

(2) Add 100 μL of 1× blocking buffer, stand at 25° C. for 2 hours, and then wash with PBST four times.

(3) Add 100 μL of 6250×, 31250×, 156250×, 781250×, 3906250×, 19531250× and 97656250× diluted rabbit immunoglobulin G (ChromPure Rabbit IgG, whole molecule, abbreviated as rabbit IgG) (diluted in a blocking buffer, 1.19 mg/ml as 1×). Stand at 25° C. for 1 hour, and then wash with PBST four times.

(4) Add 100 μL of complexes of goat anti-rabbit immunoglobulin G conjugated horseradish peroxidase (Goat anti-rabbit IgG(H+L), horseradish peroxidase conjugated) and gold nanoparticles (abbreviated as anti-rabbit IgG-HRP@gold nanoparticles), and carry out centrifugal sedimentation at 2700 rpm for 30 minutes and 60 minutes, respectively. After centrifuging, wash with PBST four times. After washing, turn the gold nanoparticle carrier by 180 degrees so that the hole of the gold nanoparticle carrier faces a bottom of a rotor of a centrifuge, centrifuge the gold nanoparticle carrier at 2700 rpm for 2 minutes to remove a liquid in the hole, and measure a full spectrum.

Table 3 shows that centrifugation for 30 minutes achieves very good results. The optimum conditions for centrifugal sedimentation are centrifugation at 2700 rpm for 30 minutes, and subsequent experiments use these as experimental parameters.

TABLE 3

| | Centrifugal sedimentation | | Centrifugal sedimentation | |
|---|---|---|---|---|
| | for 30 minutes | | for 60 minutes | |
| | | The absorbance of | | The absorbance of |
| | The peak of the | the peak of the | The peak of the | the peak of the |
| | spectral | spectral | spectral | spectral |
| | absorption curve | absorption curve | absorption curve | absorption curve |
| | after adding | after adding | after adding | after adding |
| | anti-rabbit | anti-rabbit | anti-rabbit | anti-rabbit |
| Dilution fold | IgG-HRP@gold | IgG-HRP@gold | IgG-HRP@gold | IgG-HRP@gold |
| of rabbit IgG | nanoparticles | nanoparticles | nanoparticles | nanoparticles |
| 6250X | 567 | 0.379 | 558-559 | 0.397 |
| 31250X | 565 | 0.316 | 563 | 0.331 |
| 156250X | 552 | 0.247 | 554 | 0.252 |
| 781250X | 543 | 0.205 | 545-546 | 0.218 |
| 3906250X | 542 | 0.197 | 548 | 0.219 |
| 19531250X | 542 | 0.215 | 549 | 0.229 |
| 97656250X | 539 | 0.184 | 542 | 0.189 |
| 0 | 539 | 0.181 | 543 | 0.191 |

7. Comparison of the sensitivities of experiments of localized surface plasmon resonance in the condition of centrifugal sedimentation or standing after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles):

(1) Add 100 μL of 1500 ng/cm² rabbit immunoglobulin G (ChromPure Rabbit IgG whole molecule, abbreviated as rabbit IgG) into a gold nanoparticle carrier, microwave at 60 W for 30 min, and then wash with 250 μL of PBST two times (soak for 30 seconds).

(2) Add 100 μL of 10% BSA (1×PBS), centrifuge at 2700 rpm for 30 minutes and stand for 30 minutes, and wash with 250 μL of PBST one time (soak for 30 seconds).

(3) Add biotin-conjugated goat anti-rabbit immunoglobulin G (Biotin-SP-conjugated AffiniPure Goat Anti-Rabbit IgG(H+L), abbreviated as anti-rabbit IgG-biotin) with serial dilutions of 125×, 625×, 3125×, 15625×, 78125×, 390625× and 1953125×) (1× is 1.3 mg/ml. 125×, 625×, 3125×, 15625×, 78125×, 390625× and 1953125× are 10400, 2080, 416, 83, 17, 3 and 0.6 ng/ml, respectively after serial dilution) and blank control group, centrifuge at 2700 rpm for 30 minutes and stand for 30 minutes, wash with 250 μL of PBST two times (soak for 30 seconds), and measure a spectrum.

(4) Add 100 μL of complexes of horseradish peroxidase-streptavidin and gold nanoparticles (SA-HRP@gold nanoparticles) with an absorbance of 1, centrifuge at 2700 rpm for 30 minutes and stand for 30 minutes, wash with 250 μL of PBST two times (soak for 30 seconds), and measure a spectrum.

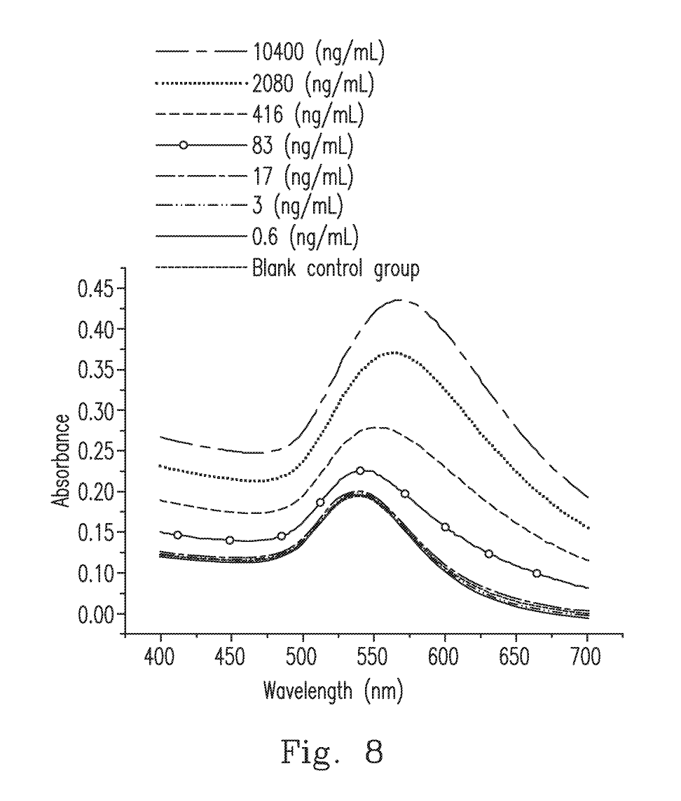

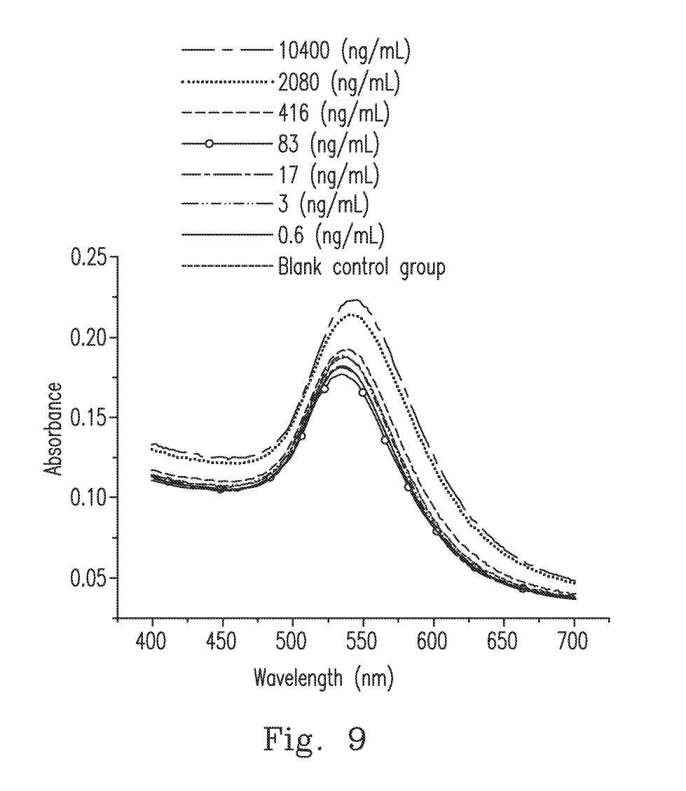

FIGS. 8-9 and Tables 4-5 show that by means of centrifuging after adding blocking buffer, biotin-conjugated goat anti-rabbit immunoglobulin G (anti-rabbit IgG-biotin) and complexes of horseradish peroxidase-streptavidin and gold nanoparticles (SA-HRP@gold nanoparticles), the sensitivity is significantly higher than by means of standing. In addition, by means of centrifuging, after adding complexes of horseradish peroxidase-streptavidin and gold nanoparticles, the absorption value is effectively amplified; by means of standing, there is no such phenomenon.

TABLE 4
By means of centrifuging

| | Measure at 550 nm | Measure at 550 nm | |
|---|---|---|---|
| | after adding | after adding | Absorption |
| Concentration | anti-rabbit | SA-HRP@gold | value |
| (ng/ml) | IgG-biotin | nanoparticles | difference |
| 10400 | 0.216 | 0.42 | 0.204 |
| 2080 | 0.196 | 0.362 | 0.166 |
| 416 | 0.181 | 0.279 | 0.098 |
| 83 | 0.177 | 0.223 | 0.046 |
| 17 | 0.173 | 0.193 | 0.02 |
| 3 | 0.171 | 0.191 | 0.02 |
| 0.6 | 0.17 | 0.189 | 0.019 |
| 0 | 0.17 | 0.188 | 0.018 |

TABLE 5
By means of standing

| | Measure at 550 nm | Measure at 550 nm | |
|---|---|---|---|
| | after adding | after adding | Absorption |
| Concentration | anti-rabbit | SA-HRP@gold | value |
| (ng/ml) | IgG-biotin | nanoparticles | difference |
| 10400 | 0.2 | 0.22 | 0.02 |
| 2080 | 0.172 | 0.21 | 0.038 |
| 416 | 0.165 | 0.184 | 0.019 |
| 83 | 0.16 | 0.165 | 0.005 |
| 17 | 0.162 | 0.169 | 0.007 |
| 3 | 0.161 | 0.176 | 0.015 |
| 0.6 | 0.162 | 0.17 | 0.008 |
| 0 | 0.161 | 0.178 | 0.017 |

8. The sensitivities of experiments of localized surface plasmon resonance in the condition of centrifugal sedimentation only after adding complexes of horseradish peroxidase-streptavidin and gold nanoparticles (SA-HRP@gold nanoparticles):

(1) Add 100 μL of 1500 ng/cm² rabbit immunoglobulin G (ChromPure Rabbit IgG whole molecule, abbreviated as rabbit IgG, formulated in coating buffer) into a gold nanoparticle carrier, microwave at 60 W for 30 min, and then wash with PBST four times.

(2) Add 100 μL of 1× blocking buffer, stand in a 25° C. incubator for 2 hours, and then wash with PBST four times.

(3) Add 100 μL of 52000, 10400, 2080, 416, 83, 17 and 3 ng/ml biotin conjugated goat anti-rabbit immunoglobulin G (Biotin-SP-conjugated AffiniPure Goat Anti-Rabbit IgG(H+L), abbreviated as anti-rabbit IgG-biotin, formulated in 1×ELISA diluent), stand at 25° C. for 2 hours, and then wash with PBST six times.

(4) Add 100 μL of complexes of horseradish peroxidase-streptavidin and gold nanoparticles (SA-HRP@gold nanoparticles), centrifuge at 2700 rpm for 30 minutes, and wash with PBST six times. After washing, turn the gold nanoparticle carrier by 180 degrees so that a hole of the gold nanoparticle carrier faces a bottom of a rotor of a centrifuge, centrifuge the gold nanoparticle carrier at 2700 rpm for 2 minutes to remove a liquid in the hole, and measure a full spectrum.

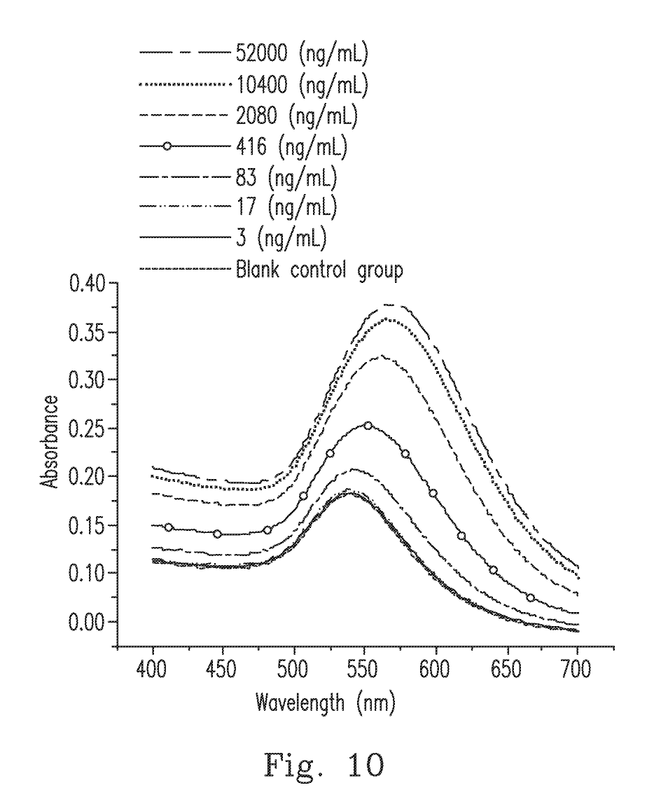

FIG. 10 and Table 6 show that absorbance at 550 nm decreases with decreasing concentration of biotin-conjugated goat anti-rabbit immunoglobulin G with a minimum detectable concentration of 3 ng/ml.

TABLE 6

| Concentration of anti-rabbit | Absorbance at 550 nm after adding |
|---|---|
| IgG-biotin (ng/ml) | SA-HRP@gold nanoparticles |
| 52000 | 0.360 |
| 10400 | 0.347 |
| 2080 | 0.317 |
| 416 | 0.253 |
| 83 | 0.204 |
| 17 | 0.180 |
| 3 | 0.177 |
| 0 | 0.175 |

9. The sensitivities of experiments of localized surface plasmon resonance in the condition of centrifugal sedimentation after adding biotin conjugated goat anti-rabbit immunoglobulin G (anti-rabbit IgG-biotin) and complexes of horseradish peroxidase-streptavidin and gold nanoparticles (SA-HRP@gold nanoparticles):

(1) Add 100 μL of 1500 ng/cm² rabbit immunoglobulin G (ChromPure Rabbit IgG whole molecule, abbreviated as rabbit IgG, formulated in coating buffer) into a gold nanoparticle carrier, microwave at 60 W for 30 min, and then wash with PBST four times.

(2) Add 100 μL of 1× blocking buffer, stand in a 25° C. incubator for 2 hours, and then wash with PBST four times.

(3) Add 100 μL of 10400, 2080, 416, 83, 17, 3 and 0.6 ng/ml biotin conjugated goat anti-rabbit immunoglobulin G (Biotin-SP-conjugated AffiniPure Goat Anti-Rabbit IgG(H+L), abbreviated as anti-rabbit IgG-biotin, formulated in 1×ELISA diluent), centrifuge at 2700 rpm for 30 minutes, and wash with PBST six times.

(4) Add 250 μL of complexes of horseradish peroxidase-streptavidin and gold nanoparticles (SA-HRP@gold nanoparticles) with an absorbance of 1, centrifuge at 2700 rpm for 30 minutes, and wash with PBST six times. After washing, turn the gold nanoparticle carrier by 180 degrees so that a hole of the gold nanoparticle carrier faces a bottom of a rotor of a centrifuge, centrifuge the gold nanoparticle carrier at 2700 rpm for 2 minutes to remove a liquid in the hole, and measure a full spectrum.

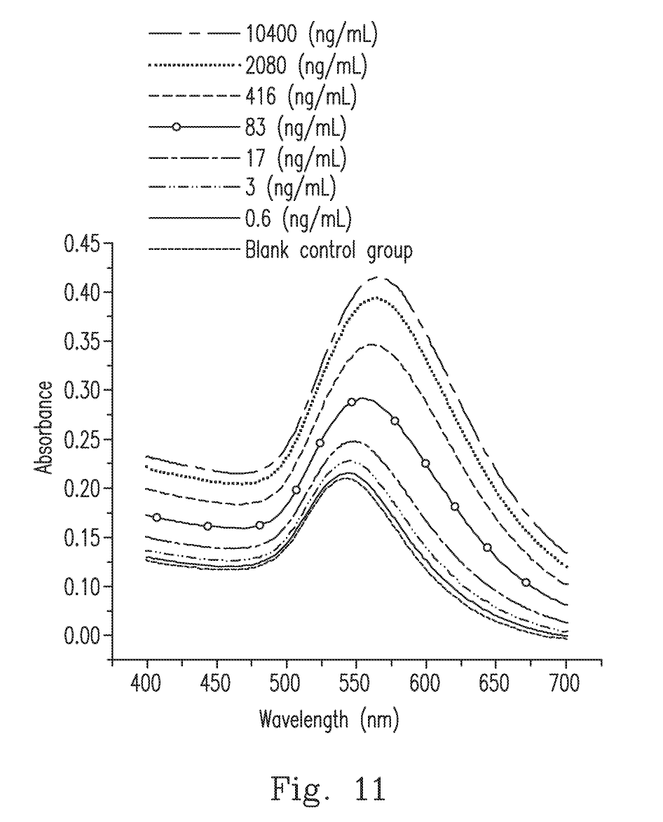

FIG. 11 and Table 7 show that the detection concentration can reach 0.6 ng/mL, indicating that also carrying out centrifugal sedimentation after adding biotin-conjugated goat anti-rabbit immunoglobulin G can improve signal sensitivity at low concentrations.

TABLE 7

| Concentration of anti-rabbit | Absorbance at 550 nm after adding |
|---|---|
| IgG-biotin (ng/ml) | SA-HRP@gold nanoparticles |
| 10400 | 0.4 |
| 2080 | 0.382 |
| 416 | 0.339 |
| 83 | 0.29 |
| 17 | 0.247 |
| 3 | 0.226 |
| 0.6 | 0.213 |
| 0 | 0.207 |

10. The sensitivities of experiments of localized surface plasmon resonance in the condition of centrifugal sedimentation after adding blocking buffer and complexes of horseradish peroxidase-streptavidin and gold nanoparticles (SA-HRP@gold nanoparticles):

(1) Add 100 μL of 1500 ng/cm² rabbit immunoglobulin G (ChromPure Rabbit IgG whole molecule, abbreviated as rabbit IgG, formulated in coating buffer) into a gold nanoparticle carrier, microwave at 60 W for 30 min, and then wash with PBST four times.

(2) Add 100 μL of 1× blocking buffer, centrifuge at 2700 rpm for 30 minutes, and then wash with PBST four times.

(3) Add 100 μL of 10400, 2080, 416, 83, 17, 3 and 0.6 ng/ml biotin conjugated goat anti-rabbit immunoglobulin G (Biotin-SP-conjugated AffiniPure Goat Anti-Rabbit IgG(H+L), abbreviated as anti-rabbit IgG-biotin, formulated in 1×ELISA diluent), stand for 2 hours, and wash with PBST six times.

(4) Add 250 μL of complexes of horseradish peroxidase-streptavidin and gold nanoparticles (SA-HRP@gold nanoparticles) with an absorbance of 1, centrifuge at 2700 rpm for 30 minutes, and wash with PBST six times. After washing, turn the gold nanoparticle carrier by 180 degrees so that a hole of the gold nanoparticle carrier faces a bottom of a rotor of a centrifuge, centrifuge the gold nanoparticle carrier at 2700 rpm for 2 minutes to remove a liquid in the hole, and measure a full spectrum.

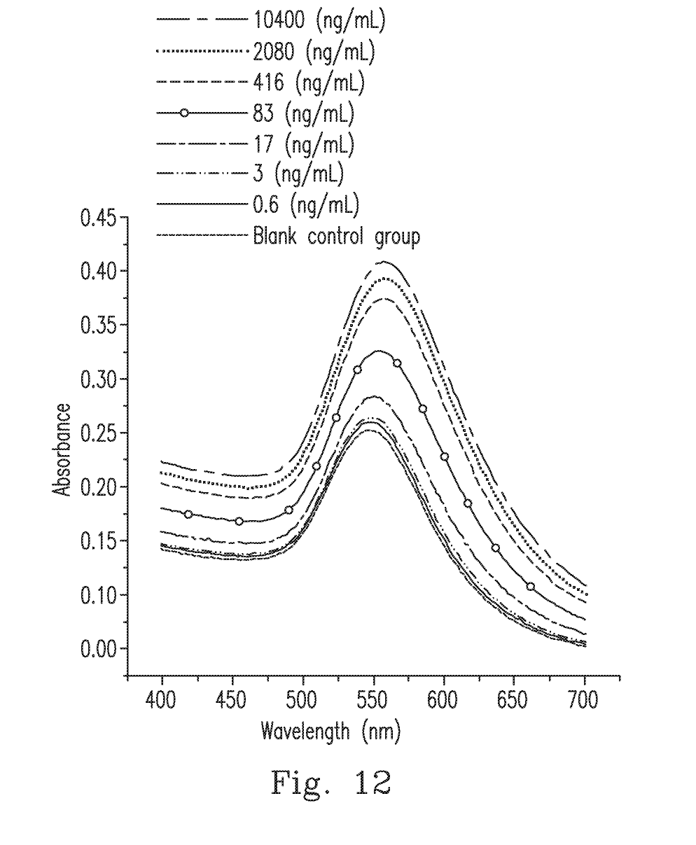

FIG. 12 and Table 8 show poor discrimination at low concentrations. It is shown that when detecting lower concentrations, biotin-conjugated goat anti-rabbit immunoglobulin G has poorer sensitivity by means of standing than by means of centrifugal sedimentation.

TABLE 8

| Concentration of anti-rabbit | Absorbance at 550 nm after adding |
|---|---|
| IgG-biotin (ng/ml) | SA-HRP@gold nanoparticles |
| 10400 | 0.4073 |
| 2080 | 0.3921 |
| 416 | 0.3726 |
| 83 | 0.3255 |
| 17 | 0.2850 |
| 3 | 0.2640 |
| 0.6 | 0.26 |
| 0 | 0.252 |

11. The sensitivities of experiments of localized surface plasmon resonance in the condition of centrifugal sedimentation after adding blocking buffer, biotin conjugated goat anti-rabbit immunoglobulin G (anti-rabbit IgG-biotin) and complexes of horseradish peroxidase-streptavidin and gold nanoparticles (SA-HRP@gold nanoparticles):

(1) Add 100 μL of 1500 ng/cm² rabbit immunoglobulin G (ChromPure Rabbit IgG whole molecule, abbreviated as rabbit IgG, formulated in coating buffer) into a gold nanoparticle carrier, microwave at 60 W for 30 min, and then wash with PBST four times.

(2) Add 100 μL of 1× blocking buffer, centrifuge at 2700 rpm for 30 minutes, and then wash with PBST four times.

(3) Add 100 μL of 10400, 2080, 416, 83, 17, 3 and 0.6 ng/ml biotin conjugated goat anti-rabbit immunoglobulin G (Biotin-SP-conjugated AffiniPure Goat Anti-Rabbit IgG(H+L), abbreviated as anti-rabbit IgG-biotin, formulated in 1×ELISA diluent), centrifuge at 2700 rpm for 30 minutes, and wash with PBST six times.

(4) Add 250 μL of complexes of horseradish peroxidase-streptavidin and gold nanoparticles (SA-HRP@gold nanoparticles) with an absorbance of 1, centrifuge at 2700 rpm for 30 minutes, and wash with PBST six times. After washing, turn the gold nanoparticle carrier by 180 degrees so that a hole of the gold nanoparticle carrier faces a bottom of a rotor of a centrifuge, centrifuge the gold nanoparticle carrier at 2700 rpm for 2 minutes to remove a liquid in the hole, and measure a full spectrum.

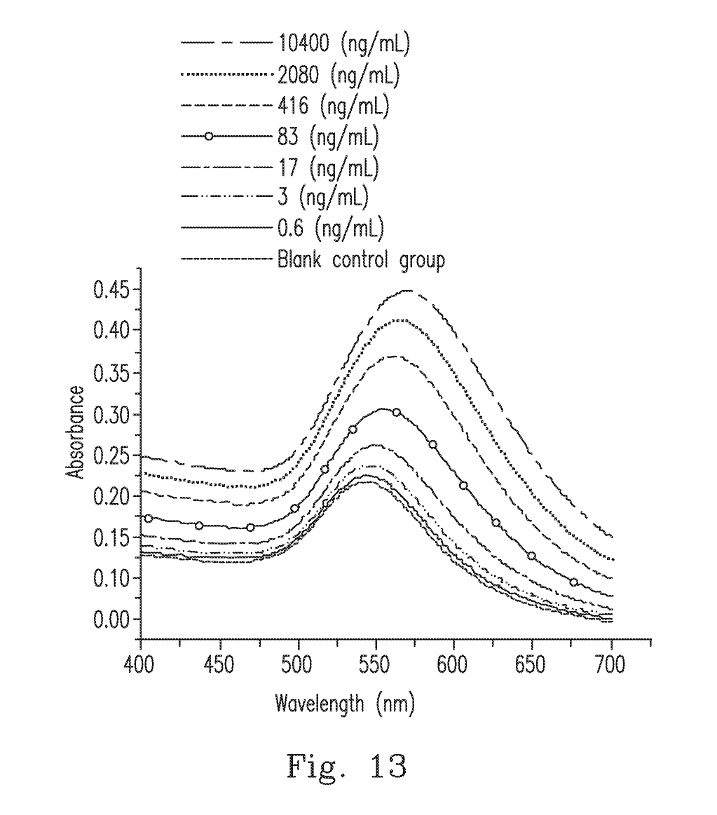

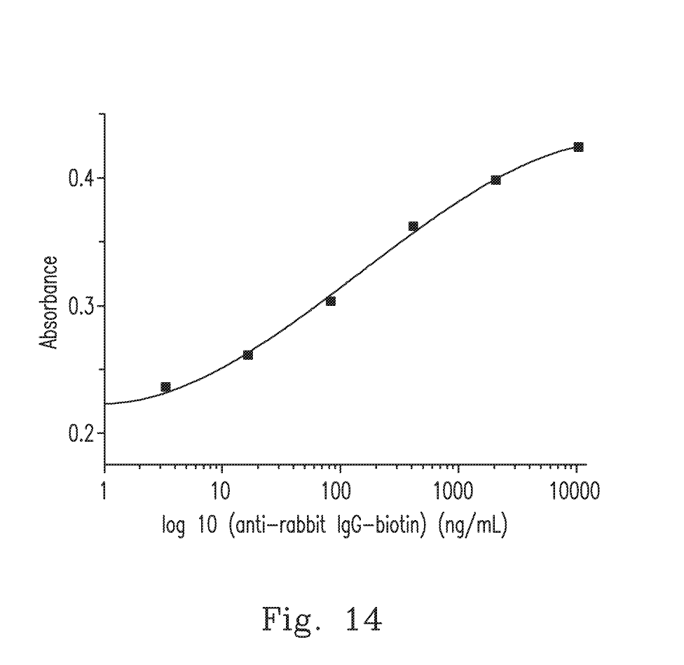

Please refer to FIGS. 13-14 and Table 9, wherein FIG. 13 shows a full spectrum measured after adding complexes of horseradish peroxidase-streptavidin and gold nanoparticles, and FIG. 14 shows a plot of an absorbance at 550 nm of the full spectrum measured after adding complexes of horseradish peroxidase-streptavidin and gold nanoparticles versus concentration of biotin conjugated goat anti-rabbit immunoglobulin G (R²=0.995). FIGS. 13-14 and Table 9 show that absorbances decrease with decreasing concentrations of biotin-conjugated goat anti-rabbit immunoglobulin G with a minimum detectable concentration of 0.6 ng/ml.

TABLE 9

| Concentration of anti-rabbit | Absorbance at 550 nm after adding |
|---|---|
| IgG-biotin (ng/ml) | SA-HRP@gold nanoparticles |
| 10400 | 0.424 |
| 2080 | 0.398 |
| 416 | 0.362 |
| 83 | 0.303 |
| 17 | 0.261 |
| 3 | 0.236 |
| 0.6 | 0.222 |
| 0 | 0.215 |

From the experimental data of experiments of localized surface plasmon resonance in the condition of centrifugal sedimentation, it can be seen that changing the traditional standing method into the centrifugal sedimentation method reduces the adsorption time, and thus reduces the reaction time to only one-third of the original, and further increases the sensitivity from 17 ng/mL to 0.6 ng/mL, nearly 30 times.

12. Detection of hepatitis C virus (abbreviated as HCV) antibodies in a patient with hepatitis C:

(1) Add 100 μL of 6000 ng/cm² hepatitis C core antigen recombinant protein (HCV core antigen recombinant protein, abbreviated as core protein, formulated in coating buffer) into a gold nanoparticle carrier, microwave at 60 W for 30 min, and then wash with PBST four times.

(2) Add 100 μL of 1× blocking buffer, centrifuge at 2700 rpm for 30 minutes, and then wash with PBST four times.

(3) Add 100 μL of 2, 10, 50 and 250× diluted patient's serum (formulated in 1× ELISA diluent), stand at 25° C. for 2 hours, and wash with PBST six times after centrifugation. In addition, add 100 μL of 2, 10, 50, 250, 1250, 6250 and 31250× diluted patient's serum (formulated in 1×ELISA diluent), centrifuge at 2700 rpm for 30 minutes, and wash with PBST six times.

(4) Add 250 μL of complexes of goat anti-human immunoglobulin G conjugated horseradish peroxidase (Peroxidase-conjugated AffiniPure Goat Anti-Human IgG) and gold nanoparticles (abbreviated as anti-human IgG-HRP@gold nanoparticles) with an absorbance of 1, centrifuge at 2700 rpm for 30 minutes, and wash with PBST six times. After washing, turn the gold nanoparticle carrier by 180 degrees so that a hole of the gold nanoparticle carrier faces a bottom of a rotor of a centrifuge, centrifuge the gold nanoparticle carrier at 2700 rpm for 2 minutes to remove a liquid in the hole, and measure a full spectrum.

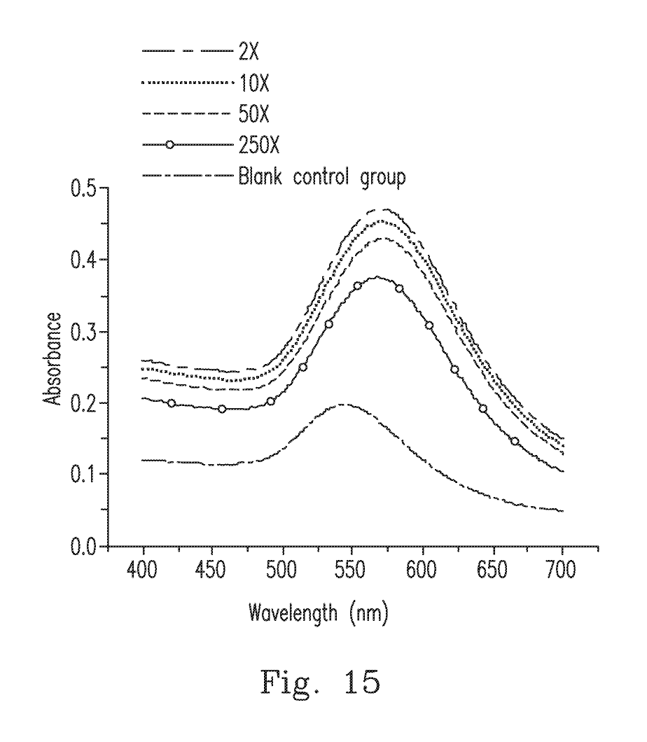

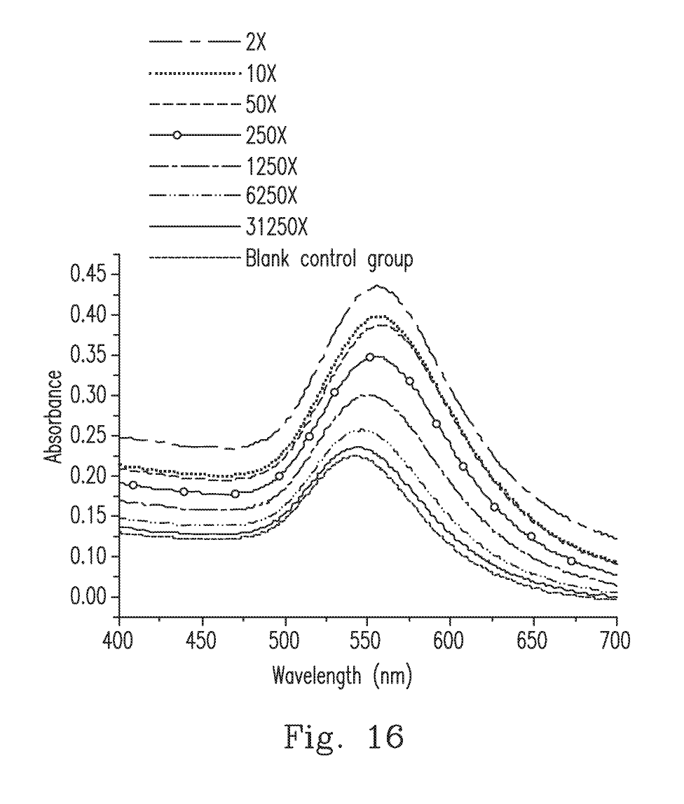

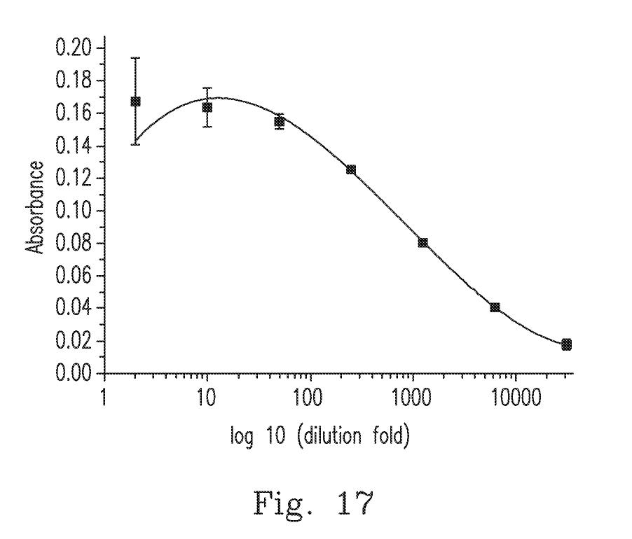

Please refer to FIGS. 15-17 and Tables 10-11, wherein FIG. 15 shows a full spectrum measured after adding complexes of goat anti-human immunoglobulin G conjugated horseradish peroxidase and gold nanoparticles in the condition of standing patient's serum, FIG. 16 shows a full spectrum measured after adding complexes of goat anti-human immunoglobulin G conjugated horseradish peroxidase and gold nanoparticles in the condition of centrifuging patient's serum, and FIG. 17 shows a plot of an absorbance at 550 nm of a full spectrum measured after adding complexes of goat anti-human immunoglobulin G conjugated horseradish peroxidase and gold nanoparticles in the condition of centrifuging patient's serum versus dilution fold of patient's serum (R²=0.998). FIGS. 15-17 and Tables 10-11 show that in the condition of centrifugal sedimentation, the absorbance decreases with the dilution fold of patient's serum, and 31250× diluted serum can be detected. In comparison to standing, it is shown that at high concentrations, centrifugal sedimentation can reduce the experimental time without affecting the sensitivity.

TABLE 10

| Dilution fold of the patient's serum (in the | |
|---|---|
| condition of standing patient's serum) | Absorbance at 550 nm |
| 2X | 0.445 |
| 10X | 0.425 |
| 50X | 0.401 |
| 250X | 0.356 |
| Blank control group | 0.196 |

TABLE 11

| Dilution fold of the patient's serum (in the | |
|---|---|
| condition of centrifugal sedimentation | |
| patient's serum) | Absorbance at 550 nm |
| 2X | 0.431 |
| 10X | 0.394 |
| 50X | 0.382 |
| 250X | 0.346 |
| 1250X | 0.300 |
| 6250X | 0.256 |
| 31250X | 0.233 |
| Blank control group | 0.221 |

13. Centrifugal Sedimentation for Enzyme-linked immunosorbent assay (ELISA) sensitivity:

(1) Primary Antibodies: Add 100 μL of 250× diluted capture antibody, i.e. anti-mouse tumor necrosis factor alpha (Anti-Mouse TNF alpha Purified (250×), diluted in coating buffer), microwave at 60 W for 30 min, and then wash with 250 μl of PBST supplemented with 0.05% Tween 20 four times.

(2) Blocking: Add 100 μL of 1× blocking buffer, stand for 2 hours in a 25° C. incubator (or centrifuged at 2700 rpm for 30 minutes), and wash four times with 250 μl of PBST supplemented with 0.05% Tween 20.

(3) Add 100 μL of 1000, 100, 10, 1, 0.1 and 0.01 pg/ml diluted mouse tumor necrosis factor alpha lyophilized standard (Mouse TNF alpha Lyophilized Standard, abbreviated as mouse TNF alpha) (diluted in diluent and blank control group add only diluent), stand for 2 hours in a 25° C. incubator (or centrifuged at 2700 rpm for 30 minutes), and washed four times with 250 μl of PBST supplemented with 0.05% Tween 20.

(4) Secondary antibody: Add 100 μL of 250× diluted detection antibody, i.e. anti-mouse tumor necrosis factor alpha-biotin (Anti-Mouse TNF alpha Biotin (250×), diluted in coating buffer), stand for 2 hours in a 25° C. incubator (or centrifuged at 2700 rpm for 30 minutes), and wash six times with 250 μl of PBST supplemented with 0.05% Tween 20.

(5) Add 100 μL of horseradish peroxidase-streptavidin (SA-HRP), stand for 30 minutes in a 25° C. incubator, wash six times with 250 μl of PBST supplemented with 0.05% Tween 20, add 100 μl of TMB, stand for 15 minutes for coloration, and finally add 50 μL of H₂SO₄ to terminate the reaction and measure a spectrum.

Please refer to Table 12. In the ELISA experiments, centrifugal sedimentation reduces the sensitivity and the OD value, which may be because centrifugal sedimentation forces the antibody to adsorb to a bottom of a carrier, causing a wall of the carrier to be unable to effectively adsorb antibody, and causing the color signal to be relatively weak. However, the experimental results of ELISA confirm once again that centrifugal sedimentation can adsorb molecules to the bottom of the carrier, and thus it is favorable for enhancing the sensitivity of localized surface plasmon resonance.

TABLE 12

| | Gold nanoparticle carrier | | Corning's COR-9018 plate | |
|---|---|---|---|---|
| | Stand diluent, | Centrifuge | Stand diluent, | Centrifuge |
| | mouse TNF | diluent, mouse | mouse TNF | diluent, mouse |
| | alpha and | TNF alpha and | alpha and | TNF alpha and |
| Concentration | secondary | secondary | secondary | secondary |
| of mouse TNF | antibody for 2 | antibody for 30 | antibody for 2 | antibody for 30 |
| alpha | hours | minutes | hours | minutes |
| 1000 | Exceed the | 1.824 | 3.496 | 2.465 |
| | measurable limit | | | |
| 100 | 0.8187 | 0.2609 | 0.9700 | 0.5271 |
| 10 | 0.1539 | 0.07256 | 0.1347 | 0.07525 |
| 1 | 0.07512 | 0.05490 | 0.03445 | 0.02321 |
| 0.1 | 0.06674 | 0.05436 | 0.02792 | 0.01726 |
| 0.01 | 0.06295 | 0.05442 | 0.02606 | 0.01989 |
| Blank control | 0.06691 | 0.05710 | 0.02292 | 0.01667 |
| group | | | | |
| Blank control | 0.06638 | 0.05931 | 0.02623 | 0.02004 |
| group | | | | |

### EMBODIMENTS

1. A sensing method, comprising steps of providing a carrier having a hole including a bottom, wherein a substrate is disposed on the bottom of the hole, a plurality of first nanoparticles are disposed on the substrate and spaced apart from each other, and the plurality of first nanoparticles generate a first spectral signal when excited; providing a first organic molecule into the hole; coating the first organic molecule in the hole; providing a second organic molecule into the hole, wherein when the second organic molecule has a first specific binding with the first organic molecule, the first spectral signal converts to a second spectral signal if the plurality of first nanoparticles are excited; providing a complex including a third organic molecule into the hole; centrifuging the carrier to subside the complex, wherein when the third organic molecule has a second specific binding with the second organic molecule, the second specific binding amplifies the second spectral signal into a third spectral signal having a specific value if the plurality of first nanoparticles are excited, wherein the first, the second and the third spectral signals are generated due to localized surface plasmon resonance; and measuring the specific value of the third spectral signal.
2. The sensing method of Embodiment 1, wherein the complex further includes a second nanoparticle coupled to the third organic molecule.
3. The sensing method of any one of Embodiments 1-2, wherein each of the first nanoparticle and the second nanoparticle includes a metal.
4. The sensing method of any one of Embodiments 1-3, wherein the metal is at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir).
5. The sensing method of any one of Embodiments 1-4, further comprising a step of: after providing the second organic molecule into the hole, centrifuging the carrier at 500-6000 rpm for 15-60 minutes to subside the second organic molecule.
6. The sensing method of any one of Embodiments 1-5, wherein the first organic molecule, the second organic molecule, and the complex are all of a liquid form.
7. The sensing method of any one of Embodiments 1-6, further comprising a step of placing the carrier in a rotor of a centrifuge and centrifuging the carrier at 500-6000 rpm for 15-60 minutes to subside the complex.
8. The sensing method of any one of Embodiments 1-7, further comprising a step of placing the carrier in a rotor of a centrifuge and centrifuging the carrier at 2000-3000 rpm for 15-60 minutes to subside the complex.
9. The sensing method of any one of Embodiments 1-8, further comprising steps of turning the carrier by 180 degrees so that the hole faces a bottom of a rotor; and centrifuging the carrier at 500-6000 rpm for 0.5-2 minutes to remove a liquid in the hole.
10. The sensing method of any one of Embodiments 1-9, further comprising steps of providing a blocking buffer into the hole after coating the first organic molecule; and centrifuging the carrier at 500-6000 rpm for 15-60 minutes to block the area where the first organic molecule is not coated.
11. A sensing method, comprising steps of providing a carrier including a hole having a bottom disposed a substrate thereat, wherein a plurality of first nanoparticles are disposed on the substrate and spaced apart from each other, and the plurality of first nanoparticles generate a spectral signal when excited; providing a plurality of first molecules into the hole; coating the first molecules in the hole; providing a plurality of complexes including a plurality of second molecules and a plurality of second nanoparticles into the hole; centrifuging the carrier to subside the complex, wherein: when the second molecules have a specific binding with the first molecules, the spectral signal generated by the plurality of first nanoparticles produces a change having a value; and when the second molecules have the specific binding with the first molecules, a coupling effect is generated between the first nanoparticles and the second nanoparticles to amplify the change of the spectral signal; and measuring the value of the change, wherein the change is produced through localized surface plasmon resonance.
12. The sensing method of Embodiments 11, wherein the centrifuging step is performed under 500-6000 rpm for 15-60 minutes.
13. The sensing method of any one of Embodiments 11-12, wherein the centrifuging step is performed under 2000-3000 rpm for 15-60 minutes.
14. The sensing method of any one of Embodiments 11-13, further comprising a step of placing the carrier in a rotor of a centrifuge and centrifuging the carrier at 500-6000 rpm for 15-60 minutes to subside the complex.
15. The sensing method of any one of Embodiments 11-14, further comprising steps of providing a blocking buffer into the hole after coating the first molecules; centrifuging the carrier at 500-6000 rpm for 15-60 minutes to block an area where the first molecules are not coated; placing the carrier by turning 180 degrees so that the hole faces a bottom of a rotor; and centrifuging the carrier at 500-6000 rpm for 0.5-2 minutes to remove a liquid in the hole.
16. The sensing method of any one of Embodiments 11-15, wherein the change is measured from a single wavelength.
17. The sensing method of any one of Embodiments 11-16, wherein the single wavelength is 550 nm.
18. A sensing method, comprising steps of providing a carrier including a hole having a bottom, wherein a plurality of spaced-part first nanoparticles are disposed on the bottom; coating a sensing molecule in the hole; providing a testing solution having a testing parameter to the hole, wherein the testing solution has a complex including a testing molecule and a second nanoparticle, and a specific binding occurs between the testing molecule and the sensing molecule; centrifuging the carrier to subside the complex; washing the hole; and measuring a synthetic spectral signal change of the first nanoparticle and the second nanoparticle according to a degree of the specific binding between the testing molecule and the sensing molecule to determine the testing parameter of the testing solution.
19. The sensing method of Embodiment 18, wherein the synthetic spectral signal change comes from localized surface plasmon resonance.
20. The sensing method of any one of Embodiments 18-19, further comprising steps of providing a blocking buffer into the hole after coating the sensing molecule; and centrifuging the carrier at 500-6000 rpm for 15-60 minutes to block an area where the sensing molecule is not coated; placing the carrier by turning 180 degrees so that the hole faces a bottom of a rotor; and centrifuging the carrier at 500-6000 rpm for 0.5-2 minutes to remove a liquid in the hole.

While the invention has been described in terms of what is presently considered to be the most practical and preferred embodiments, it is to be understood that the invention need not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A sensing method, comprising steps of:
providing a carrier having a hole including a bottom, wherein a substrate is disposed on the bottom of the hole, a plurality of first nanoparticles are disposed on the substrate and spaced apart from each other, and the plurality of first nanoparticles generate a first spectral signal when excited;
providing a first organic molecule into the hole;
coating the first organic molecule in the hole;
providing a second organic molecule into the hole, wherein when the second organic molecule has a first specific binding with the first organic molecule, the first spectral signal converts to a second spectral signal if the plurality of first nanoparticles are excited;
providing a complex including a third organic molecule into the hole;
centrifuging the carrier to subside the complex, wherein when the third organic molecule has a second specific binding with the second organic molecule, the second specific binding amplifies the second spectral signal into a third spectral signal having a specific value if the plurality of first nanoparticles are excited, wherein the first, the second and the third spectral signals are generated due to localized surface plasmon resonance; and
measuring the specific value of the third spectral signal.

2. The sensing method according to claim 1, wherein the complex further includes a second nanoparticle coupled to the third organic molecule.

3. The sensing method according to claim 2, wherein each of the first nanoparticle and the second nanoparticle includes a metal.

4. The sensing method according to claim 3, wherein the metal is at least one selected from a group consisting of gold (Au), silver (Ag), palladium (Pd), platinum (Pt), chromium (Cr), cobalt (Co), molybdenum (Mo), copper (Cu), nickel (Ni), aluminum (Al), iron (Fe), magnesium (Mg), tin (Sn), titanium (Ti), thallium (Ta) and iridium (Ir).

5. The sensing method according to claim 1, further comprising a step of:
after providing the second organic molecule into the hole, centrifuging the carrier at 500-6000 rpm for 15-60 minutes to subside the second organic molecule.

6. The sensing method according to claim 1, wherein the first organic molecule, the second organic molecule, and the complex are all of a liquid form.

7. The sensing method according to claim 1, further comprising a step of:
placing the carrier in a rotor of a centrifuge and centrifuging the carrier at 500-6000 rpm for 15-60 minutes to subside the complex.

8. The sensing method according to claim 1, further comprising a step of:
placing the carrier in a rotor of a centrifuge and centrifuging the carrier at 2000-3000 rpm for 15-60 minutes to subside the complex.

9. The sensing method according to claim 1, further comprising steps of:
turning the carrier by 180 degrees so that the hole faces a bottom of a rotor; and
centrifuging the carrier at 500-6000 rpm for 0.5-2 minutes to remove a liquid in the hole.

10. The sensing method according to claim 1, further comprising steps of:
providing a blocking buffer into the hole after coating the first organic molecule; and
centrifuging the carrier at 500-6000 rpm for 15-60 minutes to block the area where the first organic molecule is not coated.

11. A sensing method, comprising steps of:
providing a carrier including a hole having a bottom disposed a substrate thereat, wherein a plurality of first nanoparticles are disposed on the substrate and spaced apart from each other, and the plurality of first nanoparticles generate a spectral signal when excited;
providing a plurality of first molecules into the hole;
coating the first molecules in the hole;
providing a plurality of complexes including a plurality of second molecules and a plurality of second nanoparticles into the hole;
centrifuging the carrier to subside the complex, wherein:
when the second molecules have a specific binding with the first molecules, the spectral signal generated by the plurality of first nanoparticles produces a change having a value; and
when the second molecules have the specific binding with the first molecules, a coupling effect is generated between the first nanoparticles and the second nanoparticles to amplify the change of the spectral signal; and
measuring the value of the change, wherein the change is produced through localized surface plasmon resonance.

12. The sensing method according to claim 11, wherein the centrifuging step is performed under 500-6000 rpm for 15-60 minutes.

13. The sensing method according to claim 11, wherein the centrifuging step is performed under 2000-3000 rpm for 15-60 minutes.

14. The sensing method according to claim 11, further comprising a step of:
placing the carrier in a rotor of a centrifuge and centrifuging the carrier at 500-6000 rpm for 15-60 minutes to subside the complex.

15. The sensing method according to claim 11, further comprising steps of:
providing a blocking buffer into the hole after coating the first molecules;
centrifuging the carrier at 500-6000 rpm for 15-60 minutes to block an area where the first molecules are not coated;
placing the carrier by turning 180 degrees so that the hole faces a bottom of a rotor; and
centrifuging the carrier at 500-6000 rpm for 0.5-2 minutes to remove a liquid in the hole.

16. The sensing method according to claim 11, wherein the change is measured from a single wavelength.

17. The sensing method according to claim 16, wherein the single wavelength is 550 nm.

18. A sensing method, comprising steps of:
providing a carrier including a hole having a bottom, wherein a plurality of spaced apart first nanoparticles are disposed on the bottom;
coating a sensing molecule in the hole;
providing a testing solution having a testing parameter to the hole, wherein the testing solution has a complex including a testing molecule and a second nanoparticle, and a specific binding occurs between the testing molecule and the sensing molecule;
centrifuging the carrier to subside the complex;
washing the hole; and
measuring a synthetic spectral signal change of the first nanoparticle and the second nanoparticle according to a degree of the specific binding between the testing molecule and the sensing molecule to determine the testing parameter of the testing solution.

19. The sensing method according to claim 18, wherein the synthetic spectral signal change comes from localized surface plasmon resonance.

20. The sensing method according to claim 18, further comprising steps of:
providing a blocking buffer into the hole after coating the sensing molecule; and
centrifuging the carrier at 500-6000 rpm for 15-60 minutes to block an area where the sensing molecule is not coated;
placing the carrier by turning 180 degrees so that the hole faces a bottom of a rotor; and
centrifuging the carrier at 500-6000 rpm for 0.5-2 minutes to remove a liquid in the hole.
